(19) United States
(12) Patent Application Publication
(10) Pub. No.: US 2012/0245610 A1
(43) Pub. Date: Sep. 27, 2012

(54) RETRACTABLE AND REMOVABLE BLADE UNIT FOR A SCALPEL

(75) Inventors: Julius Hajgato, Ontario (CA); Lee McDonald, Ontario (CA)

(73) Assignee: Southmedic Inc., Ontario (CA)

(21) Appl. No.: 13/425,220

(22) Filed: Mar. 20, 2012

Related U.S. Application Data
(60) Provisional application No. 61/467,129, filed on Mar. 24, 2011.

(51) Int. Cl. A61B 17/3213

(52) U.S. Cl. 606/167

(57) ABSTRACT

A detachable blade cartridge for a scalpel is provided. The cartridge includes a body configured for attachment to a scalpel handle, a scalpel blade and a blade cover engaged to the body for travel along the body between a closed position covering the blade and a retracted position to expose said blade for use. The cartridge includes a releasable catch for selectively retaining the cover in the open position or the closed position. The cartridge also includes a one-way catch comprising a detent in one of the cover or the body and a recess in the other of the cover or body to restrict movement of the cover relative to the body in a distal direction past a limit position while permitting movement in an opposed proximal direction.

### CROSS-REFERENCE TO RELATED APPLICATION

This application claims the benefit of U.S. application No. 61/467,129, filed on Mar. 24, 2011. The contents of said application are incorporated herein by reference.

### FIELD OF THE INVENTION

The invention relates to scalpels for surgery and other procedures, in particular a blade unit cartridge that can be attached to a scalpel handle, that comprises a scalpel blade and a retractable blade cover to protect the blade from dulling and to protect against injury to the user.

### BACKGROUND OF THE INVENTION

The scalpel is a basic tool for surgeons. Scalpels are generally available in either fully disposable or re-usable forms. The most common form of reusable (non-disposable) scalpel includes an elongate metal handle, with a blade mount projecting from an end of the handle. The conventional type of blade mount consists of a finger projecting from an end of the handle, configured to fit into a slotted opening in a blade. A disposable surgical blade (referred to as a “sharp”) is provided, usually in a foil package, which includes a slot configured to lock onto the finger. In order to mount the blade, the user must carefully remove the blade from its packaging and clip the blade onto the finger. In order to protect the user from contact with the blade during this procedure, the blade can be handled with forceps or other handling tool. Care must be taken to avoid dropping the blade, since if the blade strikes a sterile tray or bowl, the blade can be dulled and rendered unsuitable for further use. Removal of the blade after use also presents particular risks, since at this point the blade is contaminated. Manipulation of the blade at this point presents a risk of injury with potentially serious consequences to the user's health and safety. As well, careful and deliberate manipulation of the blade takes up valuable time in the operating theatre.

Replacement of the blade is required with each new surgical procedure and in some cases the blade may be replaced during the procedure itself if it has become dulled.

In order to reduce the risk of injury, various means have been proposed to provide a removable or retractable cover for the blade. In some cases, the cover consists of a sheath that slips over the blade during installation and removal of the blade, and which may be removed when the blade is to be used. However, the act of removing or installing the cover itself presents risks. Other proposed solutions have involved a scalpel with a retractable blade. For example, U.S. Pat. No. 7,101,382 to George et al. discloses a retractable-blade scalpel with two releasable latching elements. When the scalpel blade is in an extended position, each releasable latching element is accessible for depression by finger pressure to cause retraction of the extended blade.

Another device is disclosed in U.S. Pat. No. 5,827,309 to Jolly et al., relating to a surgical scalpel having a movable blade guard that can be retracted to expose the blade and that can be extended to cover the sharp cutting edge of the blade. The guard is mounted inside the blade handle.

Systems that include a retractable guard as disclosed above generally require a specialized handle that is specially configured and adapted for use with the blade guard. In general, such systems are not adapted for use with conventional, commonly-available scalpel handles. This tends to add to the cost of such systems and requires hospitals to stock multiple handle types.

More importantly, it can be difficult to overcome the reluctance of surgeons to use a new type of handle with which they are unfamiliar. There is thus a need to provide a system for protecting a blade which avoids drawbacks of conventional removable blade covers, and which is adapted for use with a conventional scalpel handle. Such a system thereby provides an increased level of comfort and familiarity to the surgeon, and permits hospitals to continue to use their supply of existing scalpel handles.

The present applicant's prior application no. PCT/CA2010/000703 discloses a removable blade cartridge for mounting to a conventional scalpel handle, thereby addressing the above need. The present invention relates to improvements in a scalpel having a removable cartridge.

### SUMMARY OF THE INVENTION

An object of the present invention is to provide an improved scalpel having a detachable blade cartridge, wherein the blade cartridge includes a retractable cover that protects the blade during storage, installation and disposal. A further object is to provide a blade cartridge for mounting to a scalpel handle, wherein the blade cartridge includes a body which can be mounted to a scalpel handle prior to use. A scalpel blade and a blade cover are mounted to the body, wherein the cover can slide relative to the body to selectively cover or expose the blade.

In an aspect of the present invention, a blade cover is incorporated in a removable blade cartridge that can be mounted to a conventional scalpel handle. The invention further relates to a kit consisting of a scalpel handle and a removable blade cartridge.

One aspect of the invention relates to a blade cartridge for attachment to a scalpel handle of the type that includes a blade mount comprising an elongate finger which projects forwardly from an end of the handle. Preferably, the scalpel handle is a conventional non-disposable handle that conforms to ISO standard configurations. The blade cartridge comprises a body having a mount configured to releasably engage the finger, a scalpel blade attached to the body and protruding forwardly therefrom and a blade cover slideably engaged to the body. The cover is configured to be moved between a forward, protective position fully covering the blade and a retracted position wherein the blade is exposed for use and the cover at least partially covers the body.

The blade cartridge may be provided as a disposable unit, intended for one-time use. The blade cartridge may be supplied in a sterile package or alternatively in unpackaged form for autoclaving or other form of sterilizing prior to use. An advantage of the present invention is that the blade cartridge is safe for handling when it is in its pre-use configuration with the cover extended over the blade and does not have to be supplied in an individual package, thereby saving on costs as well as eliminating waste and the step of opening and disposing of the package.

The blade cartridge can accommodate various types and sizes of blades, such as various industry standard blades. These include, for example, blade size nos. 11, 12 and 22. The blade cartridge can also fit the ISO Standard Fitting features of surgical scalpels, such as the ISO small fitting feature (No. 3) and large fitting feature (No. 4), and is adaptable to accept other fitting features as these become available. Alternatively, according to another aspect, the handle may have a non-standard blade mount, with the blade cartridge being configured to attach to the non-standard mount. For example, for certain applications it may be desirable to provide a unique mounting configuration of the handle and blade cartridge.

In one aspect, the invention relates to a detachable blade cartridge comprising:
a) a body comprising an upper surface, opposed sidewalls and a mount to fasten said body to said finger,
b) a scalpel blade mounted to and projecting from a distal end of said body,
c) a blade cover coupled to and at least partially surrounding said body for travel between a distally extended closed position covering said blade and a proximally retracted open position to expose said blade for use,
d) a one-way catch comprising a detent in one of said cover or body and a recess in the other of said cover or body configured to restrict movement of said cover relative to said body in a distal direction past a limit position while permitting movement in an opposed proximal direction, and
e) a releasable catch for selectively retaining said cover in the open position or the closed position.

The releasable catch may comprise at least one user-actuated detent associated with said cover and first and second recesses within said body corresponding to said open and closed positions respectively of the cover. The user-actuated detent is associated with a release member. The detent is biased for engagement with a selected one of said recesses upon alignment therewith for selectively positioning the cover in the open or closed position and may be released from said detent by depression of said release member. The body may comprise at least one ledge comprising a ledge surface downwardly stepped from the upper surface and extending laterally outwardly from said sidewall, wherein said first and second recesses are within said ledge. In one aspect, the ledge surface faces downwardly and said user-actuated detent is biased upwardly to engage said surface from below and move upwardly into a selected one of said recesses when aligned therewith. The cartridge may comprise two of said ledge surfaces on opposing sides of said body, each with respective first and second recesses, and two of said user-actuated detents for engagement with said opposed ledge surfaces.

In another aspect, the release member for the user-actuated detent(s) may comprises a flexible segment of said cover for vertical travel relative to the cover and at least one arm depending from said segment, wherein said user-actuated detent protrudes from said arm and is urged by said flexible portion into engagement with said detent. In this aspect, the release member comprises a portion of said flexible segment configured for depression by a user to disengage said detent from a corresponding one of said recesses. The cover may comprise an upper surface and the sidewalls include a cutaway region. The flexible segment of said cover comprises a portion of an upper surface of the cover protruding over said cutaway region.

According to another aspect, the cartridge body has at least one axially-extending groove and said cover comprises at least one groove-traveller configured to slide within said groove to guide said cover along said body.

In another aspect, the user-actuated detent and recesses of the cartridges generate an audible and/or tactile signal when brought into engagement, to signal to the user that the cover is properly positioned in either of the open or closed positions.

In another aspect, the body further comprises a window opening to at least one side of said body. The mount for fastening the cartridge to the handle includes a plate embedded within said body, said plate having a slot therein configured to engage said finger. The window exposes said slot for mounting the handle to the body. In one aspect, the body is laterally flexible in the region of said window wherein lateral flexing of said body causes said plate to flex laterally to permit release of said handle from said cartridge.

According to another aspect, the invention relates to the combination of a scalpel handle and the blade cartridge as described above, supplied as a kit. The scalpel and blade cartridge may be configured for medical/surgical use, or any other use where a scalpel-type cutting implement may be usefully employed, such as for home hobbyists.

The invention will now be further described by a description of non-limiting detailed embodiments. It will be understood that the particular elements, means, components, methods and the like described herein are presented merely by way of example, and may be varied by persons skilled in the art while remaining within the scope of the invention. As well, any directional references used herein are merely for convenience of description, and do not limit the scope of the invention, which of course may be used in any orientation. In general, directional references herein are from the reference of a scalpel in a horizontal position with the sharp edge of the blade facing downwardly. Although the present specification refers to surgical uses of scalpels, it will be evident that the present invention relates to scalpels intended for any suitable use.

Ail prior art cited herein is incorporated into this application in its entirety, if and to the extent permitted. References herein to prior art are not intended as an admission in any form that such references constitute prior art for purposes of determining the validity of any of the claims of this application, nor that such art is material to the patentability of the invention.

### BRIEF DESCRIPTION OF THE DRAWINGS

FIG. 1 is a perspective view of a scalpel system according to the invention comprising a scalpel handle and blade cartridge according to the invention, in the closed position.

FIG. 2 is a side elevational view of a scalpel system with a blade cartridge according to the present invention, in the closed (protected) position.

FIG. 3 is an opposed side elevational view.

FIG. 4 is a top plan view of the scalpel of FIG. 1.

FIG. 5 is a bottom view of the scalpel system.

FIGS. 6 and 7 are side elevational views of the scalpel system, with the cover portion shown in partial transparency to show internal structure and showing an alternative handle configuration.

FIGS. 8 and 9 are perspective views, with the cover portion shown in partial transparency to show internal structure.

FIG. 10 is a perspective view of the body portion of the blade cartridge.

FIG. 11 is a cross-section of the body portion, along line 11-11 of FIG. 10.

FIG. 12 is a perspective view of the cover portion of the blade cartridge.

FIG. 13 is a cross-section of the cover portion, along line 13-13 of FIG. 12.

FIG. 14 is a perspective view of a second embodiment of the invention.

FIG. 15 is a side elevational view of the second embodiment.

FIG. 16 is a further perspective view of the second embodiment, showing a portion of the device in an enlarged view.

FIG. 17 is a further perspective view of the second embodiment, in the open position.

### DETAILED DESCRIPTION

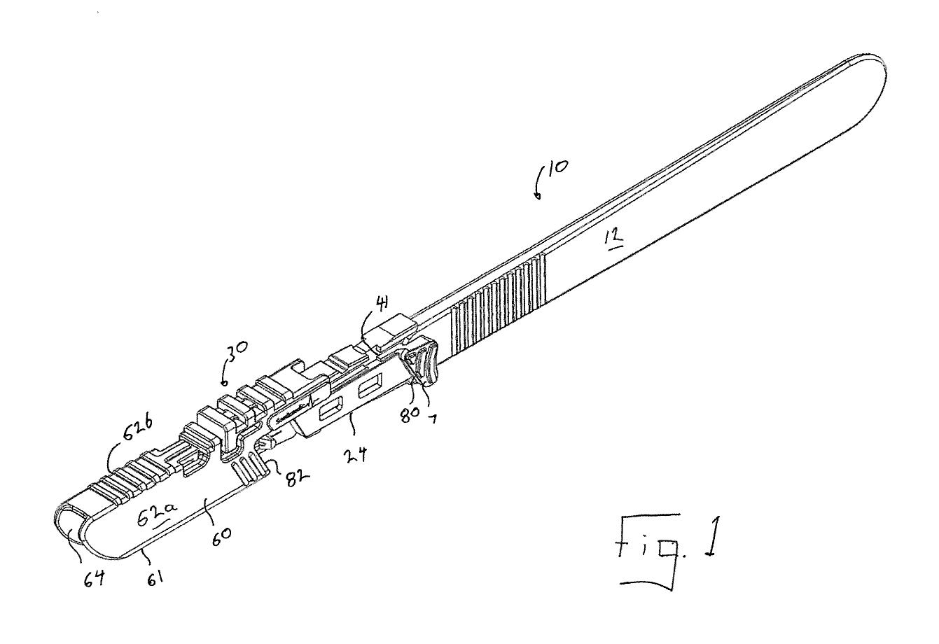

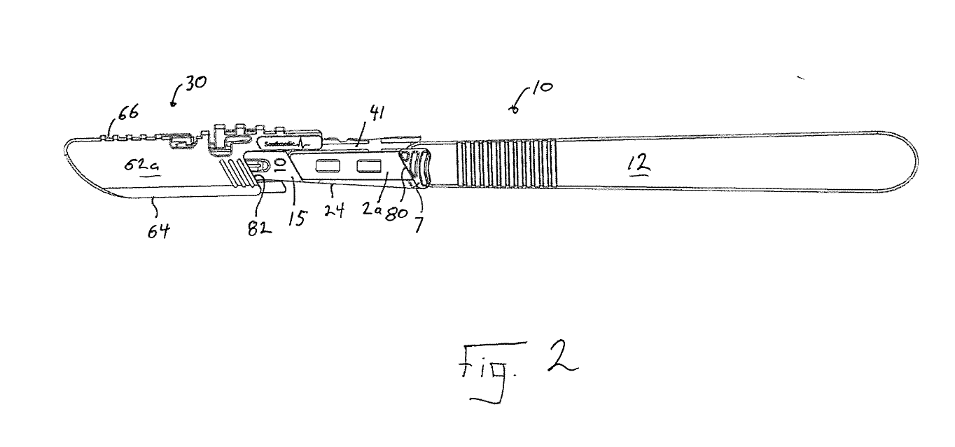

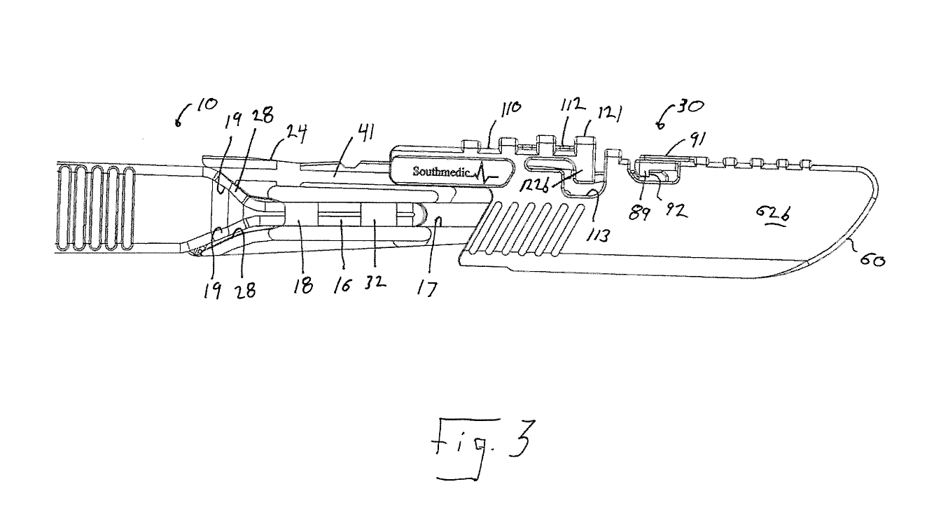

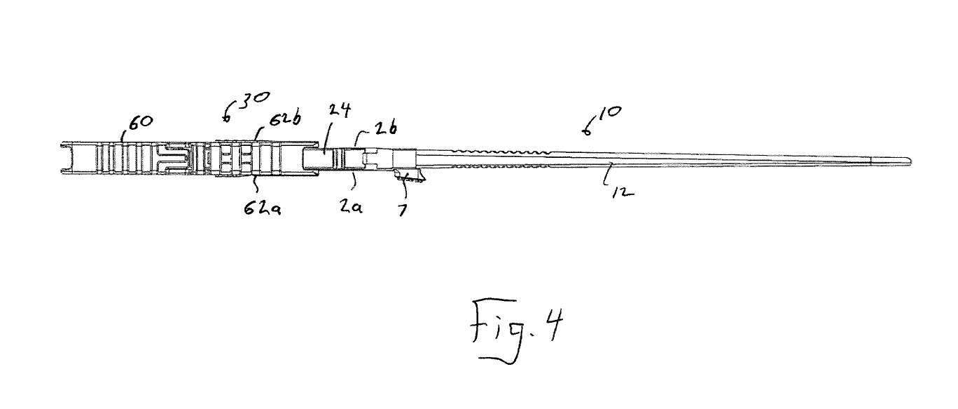

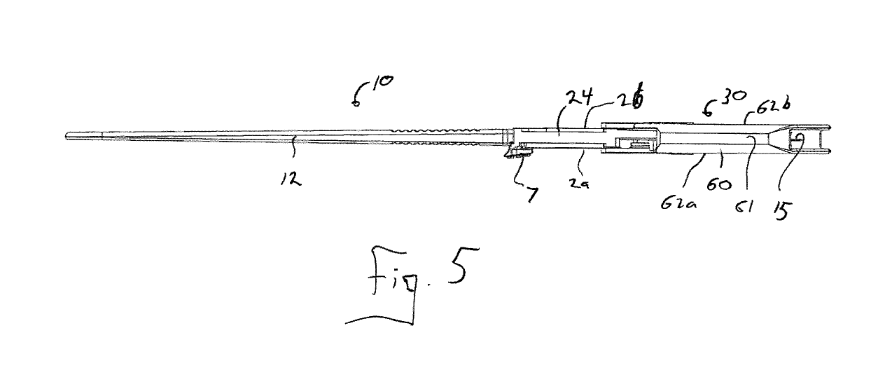

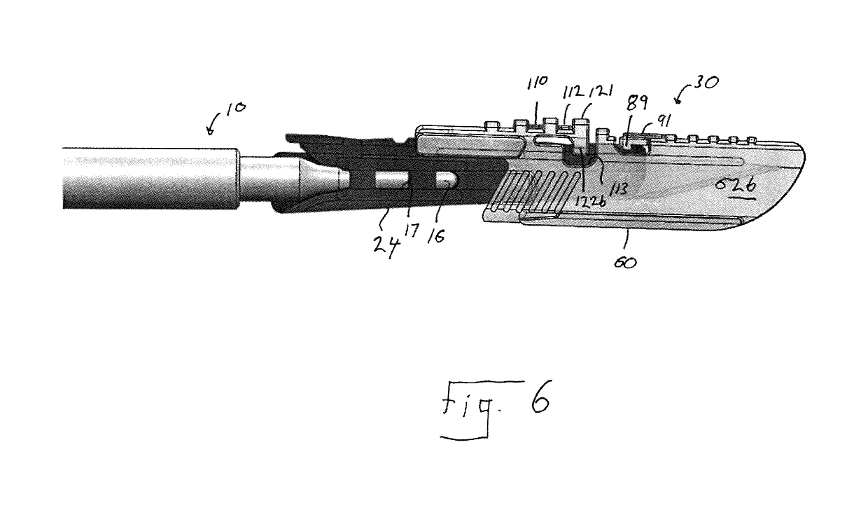

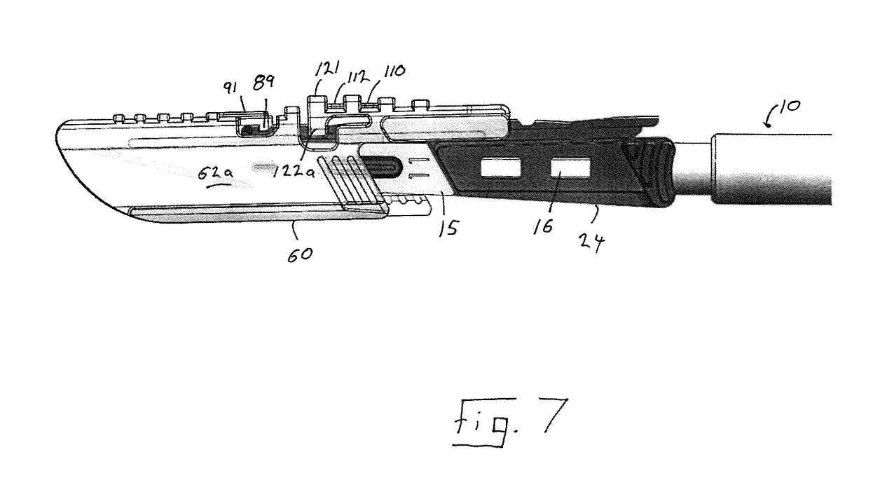

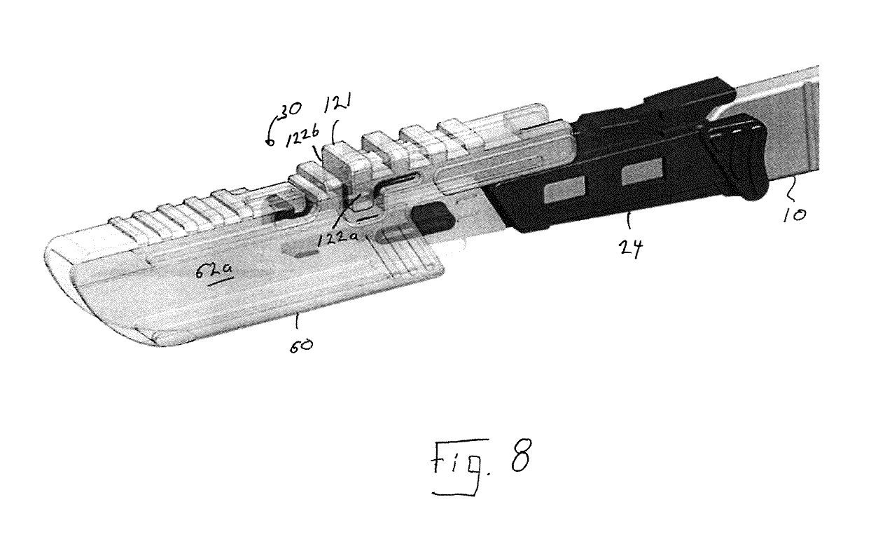

The invention relates to a scalpel system that includes a conventional scalpel handle 10, such as a Finger™ or Spectrum™ handle, as particularly shown in FIGS. 1-3. FIGS. 6 and 7 illustrate an alternative handle configuration. Handle 10 comprises an elongate hand grip portion 12 having flat lateral sides for gripping by the user's fingers. A blade-mounting finger 16 projects forwardly from the handle in a distal direction, configured to mount a blade having a conventional mounting slot during normal use of the handle. The configuration of finger 16 comprises opposing sides, one of which is substantially flat and the other of which is inwardly stepped at its proximal end where finger 16 merges with grip portion 10. The inwardly stepped portion is defined by a shoulder that normally assists in the retention of a blade to the handle or, in this case, retention of the blade cartridge to the handle.

Finger 16 is configured to normally engage a conventional surgical blade, not shown. In normal use of handle 10, a disposable blade may be clipped onto finger 16. A conventional blade of this type includes a slot configured to clip the blade to finger 16 in a manner that securely holds the blade to the handle but that may be released by flexing the blade in the region of the slot. The respective configurations of a conventional finger and blade slot meet recognized international standards (usually an ISO standard) for compatibility. Handle 10 further comprises a pair of opposed sloping shoulders 19 where the fingers join with the body portion of handle 10. Handle 10 is usually provided with ridges or other tactile features to improve the user's grip.

According to the invention, a separate disposable blade cartridge 30 is provided that is releasably attachable to handle 10. Blade cartridge 30 may be moulded from rigid plastic such as polycarbonate or polyester, which is capable of withstanding hospital autoclaving and other various sterilization methods (such as radiation, ionization, ETO). Blade cartridge 30 is radio-opaque to aid in localization in the event it is left behind postoperatively within the patient's body.

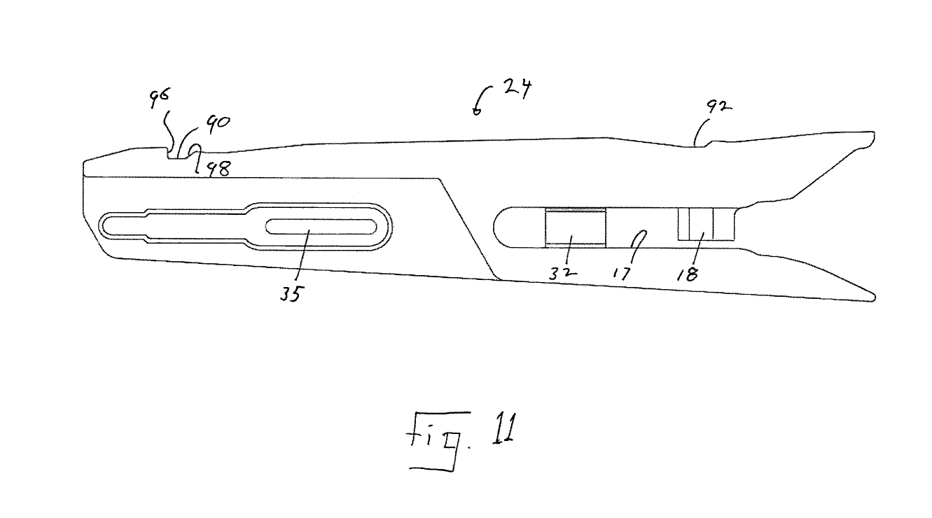

Blade cartridge 30 includes a body 24 and a sliding cover 60 which is coupled to and slides over body 24 to selectively shield the scalpel blade 15. Body 24 comprises an elongate generally flat structure, configured for mounting at its proximal end to handle 10. Body 24 comprises opposing sidewalls 2a and 2b, an upper surface 4 and an opposed lower surface 6. Body 24 further includes a mount at a proximal end region thereof to releasably fasten body 24 to handle 10. The mount comprises a shallow groove 17 recessed into sidewall 2b, configured to receive finger 16 therein to permit body 24 to clip onto finger 16. Groove 17, seen in more detail in FIG. 11, is spanned by a bridge 32 adjacent to its distal end and partially spanned by tab 18 adjacent to its proximal end to retain finger 16. Finger 16 may be inserted lengthwise into slot 17. Tab 18 flexes outwardly to allow the insertion of finger 16 into groove 17. When finger 16 is fully inserted, its distal end fits under bridge 32 and its proximal end fits under tab 18, which 18 then flexes back into its unflexed position. Tab 18 is configured to fit within the inwardly-stepped portion of finger 16 to snugly retain finger 16 within groove 17. In order to remove handle 10, the user laterally flexes blade cartridge 30 and handle 16 relative to each to cause tab 18 to flex outwardly. This then releases finger 16 from slot 17 to permit separation of blade cartridge 30 from handle 10.

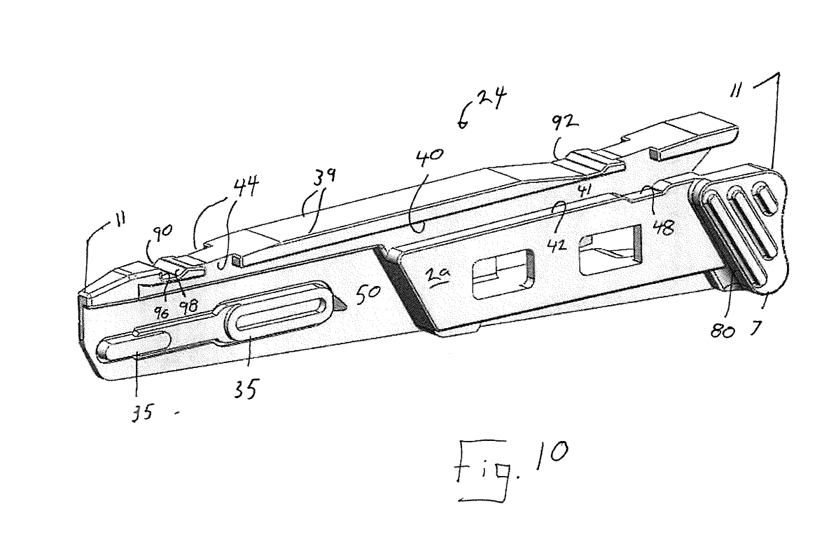

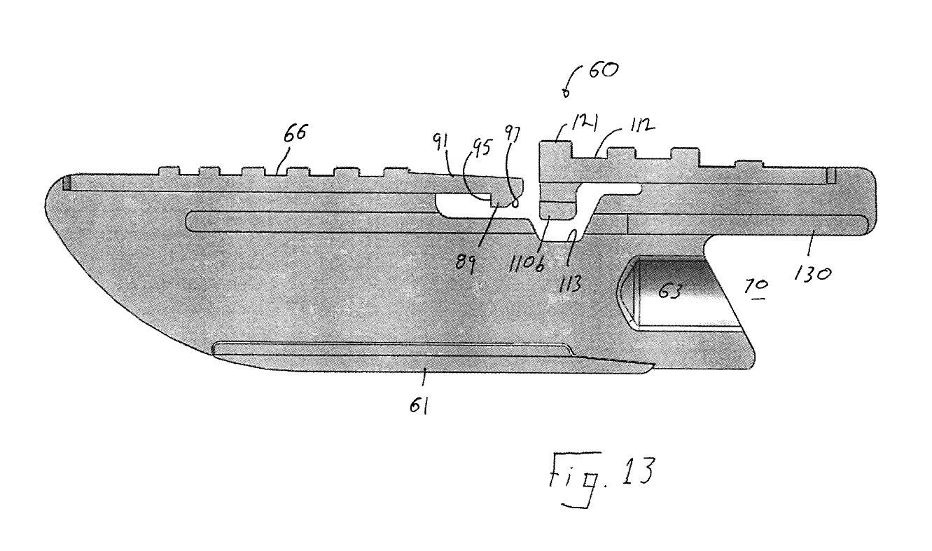

As seen in FIGS. 10 and 13, sidewalls 2a and 2b of body 24 are recessed relative to upper surface 4. As a result, upper surface 4 of body 24 overhangs sidewalls 2a and 2b, forming shelf regions 39 that project laterally outwardly from sidewalls 2a and 2b. The respective undersides of shelf regions 39 form flat overhanging ledge surfaces 40 on the respective sides of body 24 facing downwardly. At the rear (proximal) portion of body 24, ledge surfaces 40 are opposed by a shoulder 42 on either side of body 24 to form elongate opposed grooves 41 recessed into sidewalls 2a and 2b of body 24 that open laterally. Ledge surfaces 40 each provide a bearing surface to slideably engage projecting detents 110 of blade cover 60, as will be described below, for travel between an open position to expose surgical blade 15 and closed positions to cover blade 15. A first recess 44 extends into each of ledges 40 adjacent the distal end thereof. Respective recesses 44 are opposed to each other, one within each of ledges 40, and penetrate entirely through shelf regions 39 to upper surface 4. However, it will be seen that recesses 44 need not extend clear through shelves 39. A pair of second recesses 46 within shelf regions 39 are provided adjacent to the proximal end of ledge 40. A further pair of lower recesses 48 extend downwardly into shoulder 42 at a location directly opposed to each of second recesses 46.

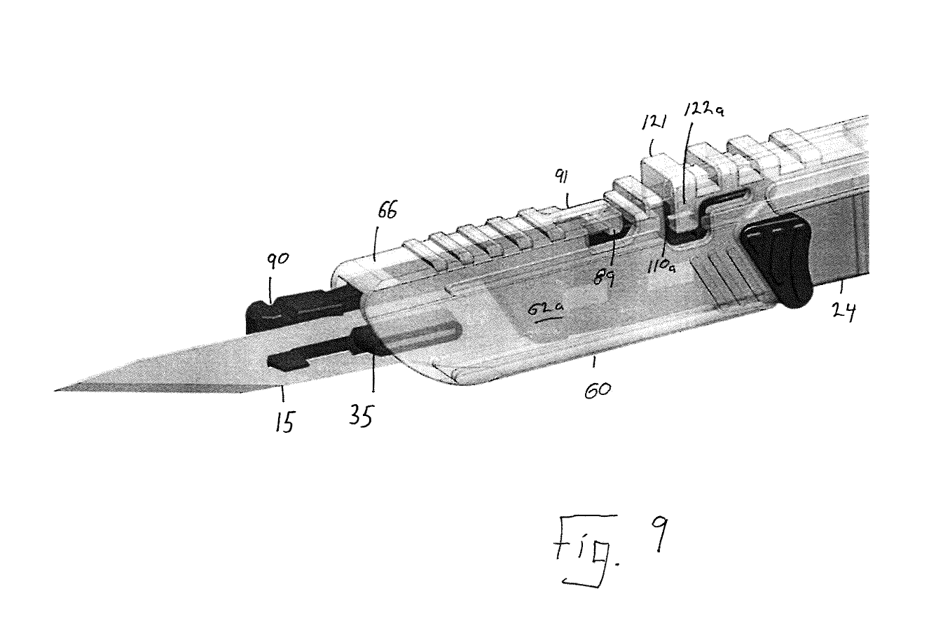

The distal end of body 24 includes a recessed portion of sidewall 2a. Surgical blade 15 is mounted to sidewall 2a within this recessed portion. Blade 15 can be permanently fixed to body 24 by melt-fastening or other permanent fastening means and protrudes forwardly therefrom. Alternatively, as seen in FIG. 9, blade 15 can be fastened by mechanical fastening means such as one or more bosses 35 projecting from body 24 that engage a mounting slot in a surgical blade 15 in a permanent fashion. Blade 15 can be fused to boss(es) 35. Blade 15 can comprise a wide range of blade configurations, depending on the desired use of the scalpel, including most conventional surgical blades or a blade configured for a hobby or other use. Depending on the size and configuration of the selected blade, the overall configuration of the blade cartridge can be scaled up or down, or otherwise adapted to accommodate the configuration of the selected blade. Persons skilled in the art will appreciate how to modify the configuration and size of the disclosed blade cartridge to suit essentially any desired scalpel.

Button 7, which protrudes laterally from one side of body 24, includes a contact surface 80 configured to contact a corresponding face 82 of the blade cover 60 when in the open position to prevent over-retraction of cover 60.

It will be seen that with modifications, body 24 may be configured to mount to a scalpel handle having a finger with a non-standard configuration or a configuration which differs from that described herein. It is foreseen that mounting systems other than that described and depicted herein may by employed for certain scalpels and other cutting implements, and in most such cases it would require only a routine modification to adapt the invention to mount to such a handle.

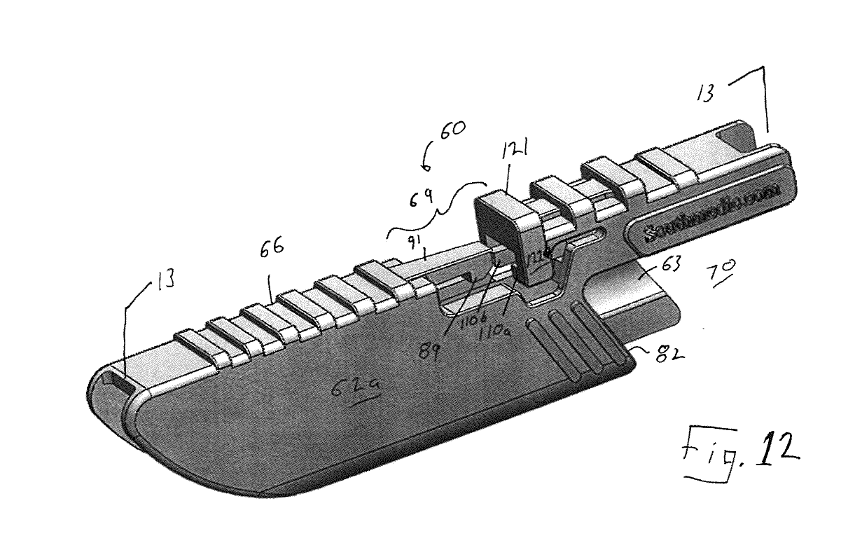

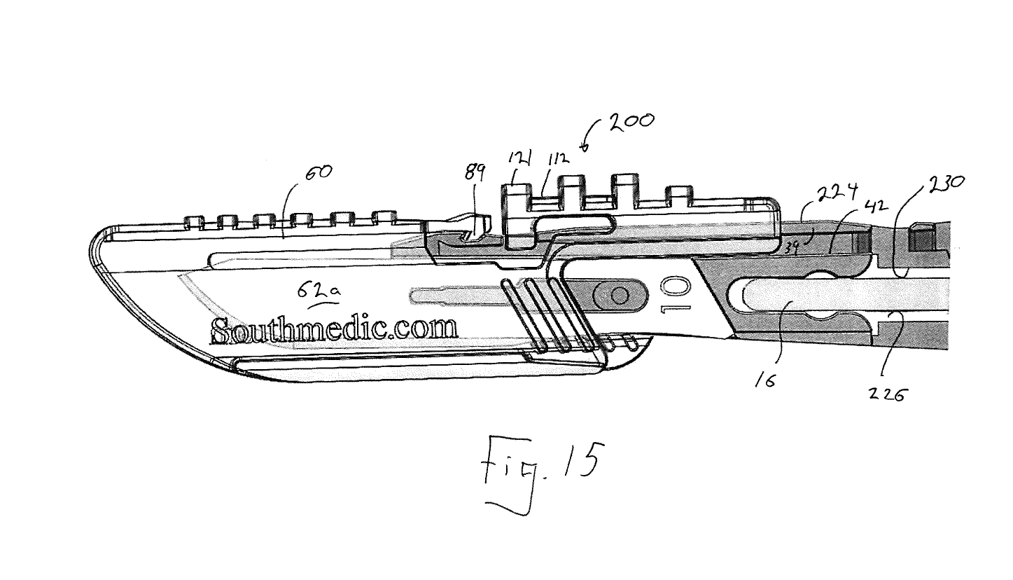

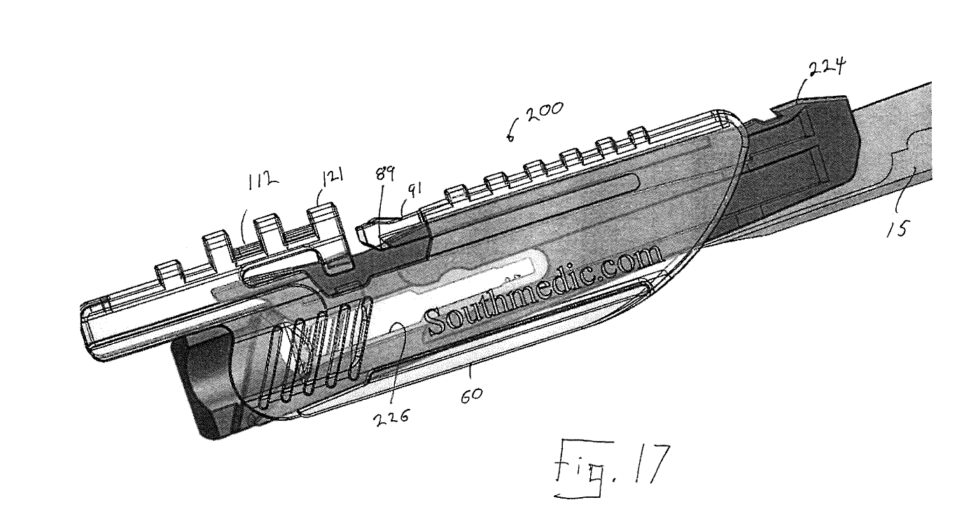

As seen in FIGS. 12-17, blade cover 60 is configured to slide axially along body 24. Cover 60 comprises an open-ended sheath that partially or fully encircles body 24 and couples to body 24 in an essentially inseparable fashion. Cover 60 is composed of spaced apart sidewalls 62a and 62b, the outside surfaces of which may include small protrusions or other features to engage the user's grip. The protrusions may be rubberized if desired. The inside surfaces of sidewalls 62a and b face sidewalls 2a and b of body 24. Cover 60 includes an upper surface 66 spanning sidewalls 62a and 62b. Upper surface 66 includes transverse ribs to improve grip. A bottom wall 61 spans sidewalls 62a and 62b to enclose the lower portion of cover 60. Blade cover 60 includes a rounded tip 64 which is open to the front to permit blade 15 to project outwardly for use when cover 60 is retracted. The inside surface of sidewall 62a includes a concave region 63, having a semi-circular cross-section, that is configured to accommodate and slide over a scalpel handle having a rounded portion with a similar cross-section.

Cover 60 is configured to shield blade 15 when extended distally relative to body 24; it will be seen that the configuration of cover 60 may vary depending on the size and configuration of blade 15 in order to fully shield blade 15. In the first embodiment described herein, blade 15 is a conventional single-edged triangular scalpel blade. Cover 60 slides axially along body 24 to travel between a storage position wherein blade 15 is covered and a retracted position.

Sidewalls 62 of cover 60 each include a proximal cut-away region 70 at the proximal end thereof, configured to expose slot 17 when cover 60 is extended to cover blade 15. This arrangement ensures that blade cartridge 30 is in a configuration to be engaged to handle 10 or disengaged therefrom only when cover 60 is extended and blade 15 is covered, thereby permitting safe handling of the device when attaching or detaching cartridge 30 to a scalpel handle 10.

Cover 60 has a gap 69 within its upper surface 66 which exposes a portion of body 24. Gap 69 is continuous with cutaway regions 113 within sidewalls 62a and b. As discussed below, these cutaway regions are configured to accommodate and permit movement of flexible portions of the cover and associated arms 122, to permit these portions to flex downwardly.

In the closed position, wherein cover 60 is extended in the distal direction, blade 15 is fully covered such that the cartridge can be safely transported, stored and handled by the user. During normal use, an inadvertent retractive force will not dislodge cover 60 from this covered position. Cover 60 can be retracted only with a deliberate force to expose blade 15, as described below. When the device is in the retracted position, blade 15 protrudes through open end 62 of cover 60 and the scalpel may be used in a conventional fashion. Rearward travel of cover 60 past the retracted (use) position is limited by contact between shoulder 82 of cover 60 and stopper 80 which prevents over-retraction of cover 60.

As seen in FIGS. 12 and 13, cover 60 includes detents that engage recesses 44, 48 and 90 in body 24 (seen in FIG. 10) to selectively retain cover 60 in its closed or open positions. In the embodiment described herein, these detents are incorporated into the cover and the recesses are within the body. However, this configuration could be modified wherein one or more of the detent/recess components are reversed in their relative positions.

Cartridge 30 includes a one-way catch that is configured to restrict movement of cover 60 relative to body 24 in a distal direction past a limit position while permitting movement in an opposed proximal direction. For this purpose, cover 60 incorporates a detent 89 located adjacent its distal end that projects downwardly from the end of a flexible, resilient arm 91. Arm 91 extends rearwardly from the forward (distal) edge of gap 69 and partially bridges this gap. Detent 89 is configured to engage a recess 90 which is within the upper surface body 24 and adjacent to the distal end of body 24. Recess 90 comprises a transverse groove spanning the upper surface of body 24, having opposed distal and proximal sidewalls and essentially the same cross sectional configuration as detent 89. Arm 91 urges detent 89 downwardly to engage recess 90 when aligned therewith. Detent 89 provides a one-way stop for cover 60. When detent 89 is engaged within recess 90, cover 60 is effectively prevented from sliding along body 24 in the distal direction, thereby preventing inadvertent separation of cover 60 from body 24. For this purpose, the respective distal faces 95 and 96 of detent 89 and recess 90 are configured to limit distal travel of cover 60 along body 24 past a predetermined “limit” position when these surfaces are in contact. In order to provide a secure and essentially non-releasable catch, faces 95 and 96 are essentially flat and either vertical or sloping upwardly and rearwardly such that when in contact and mutual engagement with each other, further distal travel of cover 60 is highly resisted. As well, arm 91 is relatively short and stiff to minimize the risk that it will flex upwardly to inadvertently release detent 89 from recess 90. The opposed, proximal, faces 97 and 98 respectively of detent 89 and recess 90 slope and/or curve in the proximal direction, wherein sliding cover 60 in a proximal direction to expose blade 15 easily urges detent 89 upwardly to be released from recess 90 as arm 91 is flexed upwardly. When detent 89 is released from recess 90, cover 60 may freely slide along body 24.

Upper surface 66 of cover 60 further comprises a recessed region 92 adjacent to the proximal end thereof. Region 92 is configured to receive detent 89 when the cover is locked in the open position. This permits arm 91 to depress into a position that is essentially flush with surface 66 to prevent hindrance to a user when gripping the scalpel.

Upper surface 66 of cover 60 includes an unsupported segment 112 that projects forwardly towards gap 69 and towards arm 91. Segment 112 is defined by cutaway portions 113 within sidewalls 62a and b of cover 60 whereby it may freely flex vertically independently of sidewalls 62. The free end of segment 112 terminates in an upwardly-projecting tab 121 and downwardly-projecting arms 122a and b. Cutaway portions 113 dip downwardly to accommodate arms 122, with sufficient space being provided beneath arms 122 to accommodate their downward movement when flexed downwardly to release detents 110. Arms 122a and b each terminate at their lower edges with inwardly-projecting detents 110a and 110b respectively. Detents 110 are configured to contact and slide along ledge surfaces 40. Ledge surfaces 40 thus effectively form a track for detents 110 whereby detents 110 slide along these surfaces as the cover is retracted or extended.

At the rear portion of body 24, detents 110a and b enter into and slide within grooves 41. Arms 122 are configured to position detents 110 to be in contact with and bear against ledge surfaces 40. When detents 110 are in contact with ledge surfaces 40, segment 112 is flexed upwardly to urge detents 110 upwardly to bear against these surfaces. As a result, when detents 110 are brought into alignment with first (distal) recesses 44, detents 110 move upwardly and enter into recesses 44 to lock cover 60 in the closed position. Cover 60 remains locked in this position until detents 110 are released by a downward force on tab 121. When cover 60 is locked in the closed position, detent 89 is also engaged within recess 90 to prevent further forward movement of cover 60 even if detents 110 are released. In this fashion, cover 60 is prevented from sliding forwardly off body 24. Detent 89 can only be released from recess 60 by a rearward movement of cover 90 when detents 110 are released. Cover 60 may thus be unlocked from the closed position by pressing tab 121 downwardly and urging the cover rearwardly to slide it into the open position to expose blade 15.

In a fashion similar to the above, when cover 60 is slid rearwardly, detents 110 are brought into alignment with second (rear) recesses 46 when cover 60 is located in the appropriate position to fully expose blade 15. At this point, upward bias exerted by segment 112 urges detents 110 upwardly to lock into rear recesses 46. Detents 110 may be released from the respective recesses by depressing segment 112 downwardly by pressing against tab 121. At the proximal end of body 24, lower recesses 48 provide spaces to receive respective detents 110 as these are pushed downwardly to release them from recesses 46. At the distal end of body 24, detents 110 are unopposed when urged downwardly out of recesses 44, and can thus be freely released when segment 112 is urged downwardly.

Preferably, an audible and/or tactile click is generated when detents 110 move into either of the front or rear recesses, to clearly indicate that to the user that the cover is locked into the open or closed position.

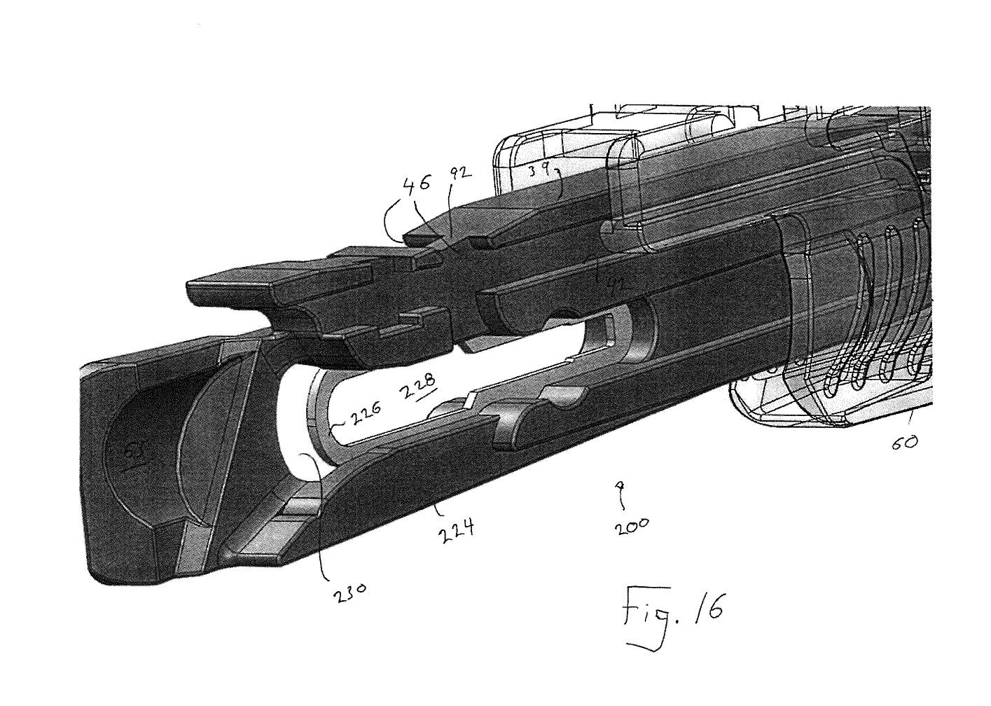

As seen in FIGS. 13 and 16, cover 60 has ribs 130 protruding inwardly from respective inside faces of sidewalls 62a and b. Ribs 130 are configured to slide within respective grooves 41 to guide cover 60 along body 24 during extension or retraction.

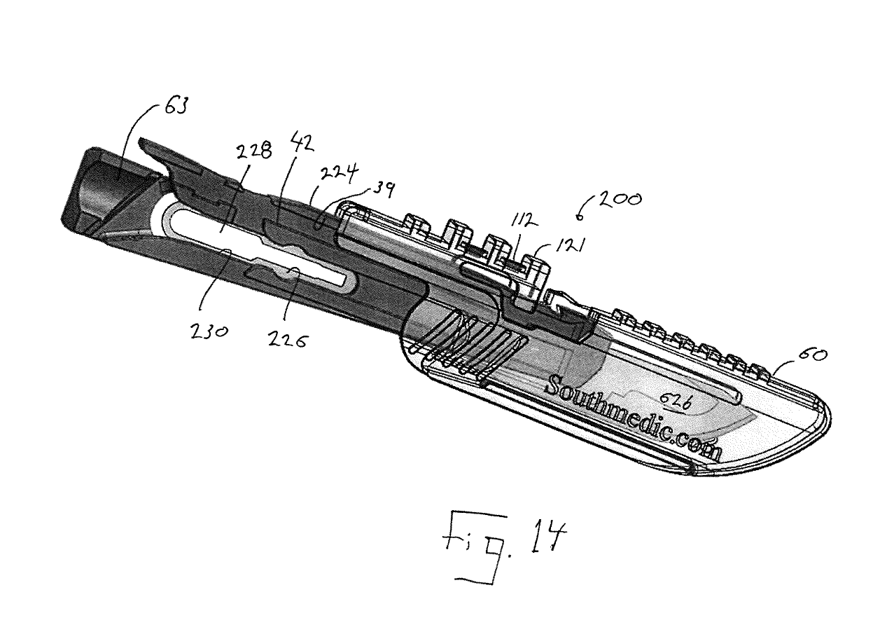

In an alternative embodiment, features of which are shown in FIGS. 14-17, a blade cartridge 200 differs from the first embodiment in the mount for engaging the body to the handle 10. In this embodiment, body 224 includes a rigid insert 226, which may be stainless steel, comprising a flat plate having a slot 228 configured to clip onto finger 16. Insert 226 is integrally moulded into body 224. Slot 228 has the same configuration as a conventional scalpel blade with a narrow distal region and a wide proximal region. This configuration permits the user to snap cartridge 200 onto the handle or release cartridge 200 by inserting finger 16 essentially longitudinally into slot 226, and releasing cartridge 200 with a lateral force and retraction of handle 10 relative to cartridge 200. Insert 226 is retained within the margins of a window 230 within body 224 that exposes the lateral sides of insert 226 to permit access to slot 228.

Body 224 has a region that is less resistant to lateral flex where shelf regions 39 and shoulders 42 are absent resulting in less structure within body 224 that resists such flex. A user may flex body 224 in this region by applying a lateral force on button 7. A retractive force, away from the midline of body 224, will cause insert 226 to flex, which when sufficiently flexed will permit release of finger 16 from slot 228 to detach cartridge 200 from handle 10.

It will be seen that the cartridges described herein can be scaled up or down in size, and the configuration of the respective components thereof altered, to accommodate different sizes and configurations of blades. As well, different materials can be employed to serve various requirements and other alterations may be made to the embodiments described herein.

A scalpel according to the present invention may be supplied to the user as a kit which includes a variety of blade cartridges with a variety of blade sizes and types. Such a kit may include one or more handles.

Although the present invention has been described in part by way of a detailed description of embodiments thereof, it will be seen by those skilled in the relevant art that the invention is not limited to the particular embodiments described above. Rather, the full scope of the present invention is defined by the present patent specification as a whole, including the claims, which should be given the widest interpretation permissible by law.

1. A cartridge for attachment to a scalpel handle of the type comprising a hand grip and a blade-mounting finger projecting from said hand grip, said cartridge comprising:
a) a body comprising an upper surface, opposed sidewalls and a mount to fasten said body to said finger,
b) a scalpel blade mounted to and projecting from a distal end of said body,
c) a blade cover coupled to and at least partially surrounding said body for travel between a distally extended closed position covering said blade and a proximally retracted open position to expose said blade for use,
d) a one-way catch comprising a detent in one of said cover or body and a recess in the other of said cover or body configured to restrict movement of said cover relative to said body in a distal direction past a limit position while permitting movement in an opposed proximal direction, and
e) a user-actuated releasable catch for selectively retaining said cover in the open position or the closed position.

2. The cartridge of claim 1 wherein said releasable catch comprises at least one user-actuated detent associated with said cover and first and second recesses within said body corresponding to said open and closed positions respectively of the cover, said user-actuated detent being associated with a release member wherein said detent is biased for engagement with a selected one of said recesses upon alignment therewith for selectively positioning the cover in the open or closed position and may be released from said detent by depression of said release member.

3. The cartridge of claim 2 wherein said body comprises at least one ledge comprising a ledge surface downwardly stepped from the upper surface and extending laterally outwardly from said sidewall, wherein said first and second recesses are within said ledge.

4. The cartridge of claim 3 wherein said ledge surface faces downwardly and said user-actuated detent is biased upwardly to engage said surface from below and move upwardly into a selected one of said recesses when aligned therewith.

5. The cartridge of claim 3 comprising two of said ledge surfaces on opposing sides of said body and two of said user-actuated detents for engagement with said opposed ledge surfaces.

6. The cartridge of claim 4 wherein release member comprises a flexible segment of said cover for vertical travel relative to the cover and at least one arm depending from said segment, wherein said user-actuated detent protrudes from said arm and is urged by said flexible portion into engagement with said detent, said release member comprising a portion of said flexible segment configured for depression by a user to disengage said detent from a corresponding one of said recesses.

7. The cartridge of claim 6 comprising two of said ledge surfaces on opposing sides of said body and two of said arms and user-actuated detents on opposing sides of said cover for engagement with said opposed ledge surfaces.

8. The cartridge of claim 6 wherein said cover comprises an upper surface and sidewalls, said sidewalls having a cutaway region, and said segment of said cover comprises a portion of an upper surface of the cover protruding over said cutaway region.

9. The cartridge of claim 7 wherein said ledges comprise opposed portions of the upper surface of said body that project laterally from the sidewalls of the body, said ledge surfaces comprising the undersides of said ledges.

10. The cartridge of claim 1 wherein said body comprises at least one axially-extending groove and said cover comprises at least one groove-traveller configured to slide within said groove to guide said cover along said body.

11. The cartridge of claim 2 wherein said user-actuated detent and recesses are configured to generate an audible and/or tactile signal when brought into engagement.

12. The cartridge of claim 1 wherein said body further comprises a window opening to at least one side of said body, said mount comprising a plate embedded within said body, said plate having a slot therein configured to engage said finger, wherein said window exposes said slot for mounting the handle to the body.

13. The cartridge of claim 1 wherein said mount comprises a groove recessed into one side of said body, and at least one resilient tab at least partially spanning said groove to retain said finger within said groove.

14. The cartridge of claim 12 wherein said body is laterally flexible in the region of said window wherein lateral flexing of said body causes said plate to flex laterally to permit decoupling of said handle from said cartridge.

15. The cartridge of claim 1 wherein said one-way catch comprises a detent in said cover and a recess in said body.

16. The cartridge of claim 15 wherein said cover comprises a flexible arm and said detent projects from said flexible arm, wherein said detent and said recess comprise first and second engagement surfaces configured to contact each other when said detent is within said recess to prevent distal travel of the cover.

17. A cartridge for attachment to a scalpel handle of the type comprising a hand grip and a finger projecting from said hand grip configured to directly mount a scalpel blade to the handle, said cartridge comprising:
a) a body comprising a mount to fasten said body to said finger, an upper surface, opposed sidewalls, at least one ledge comprising a downwardly-facing ledge surface extending laterally outwardly from said sidewall, and first and second recesses within said ledge,
b) a scalpel blade mounted to and projecting from a distal end of said body,
c) a blade cover coupled to and at least partially surrounding said body for travel between a distally extended position covering said blade and a proximally retracted position to expose said blade for use, said blade cover having a flexible segment and at least one arm extending downwardly from said flexible segment for vertical travel relative to said cover, said arm having a releasable detent protruding therefrom configured to fit within said first and second recess, said flexible segment for biasing said releasable detent upwardly into engagement with said ledge surfaces and into said recesses when aligned therewith to lock said cover into an position relative to the body by engaging said first recess or a closed position by engaging said second recess, said releasable detent being releasable from said recesses by depression of said segment, and
d) a one-way catch comprising a detent in one of said cover and body and a recess in the other of said cover and body configured to restrict movement of said cover relative to said body in a distal direction past a limit position while permitting movement in an opposed proximal direction.

18. The cartridge of claim 15 comprising two of said ledges and ledge surfaces on opposing sides of said body, each of said ledges comprising said first and second recesses therein, said cover comprising two of said arms and releasable detents on opposing sides thereof for engagement with said opposed ledge surfaces.

19. A kit of parts comprising at least one scalpel handle and at least one cartridge as defined in claim 1.

20. A scalpel comprising a scalpel handle and a cartridge as defined in claim 1.

21. A kit of parts comprising at least one scalpel handle and at least one cartridge as defined in claim 17.

22. A scalpel comprising a scalpel handle and a cartridge as defined in claim 17.
